(19) United States
(12) Patent Application Publication
(10) Pub. No.: US 2010/0286519 A1
(43) Pub. Date: Nov. 11, 2010

(54) ULTRASOUND SYSTEM AND METHOD TO AUTOMATICALLY IDENTIFY AND TREAT ADIPOSE TISSUE

(75) Inventors: WARREN LEE, NISKAYUNA, NY (US); DHIRAJ ARORA, NISKAYUNA, NY (US); CYNTHIA ELIZABETH LANDBERG DAVIS, NISKAYUNA, NY (US); YING FAN, NISKAYUNA, NY (US); CHRISTOPHER ROBERT HAZARD, NISKAYUNA, NY (US); KENNETH WAYNE RIGBY, CLIFTON PARK, NY (US); LOWELL SCOTT SMITH, NISKAYUNA, NY (US); KAI ERIK THOMENIUS, CLIFTON PARK, NY (US)

Correspondence Address: DEAN D. SMALL;THE SMALL PATENT LAW GROUP LLP, 225 S. MERAMEC, STE. 725T, ST. LOUIS, MO, 63105, US

(73) Assignee: GENERAL ELECTRIC COMPANY, SCHENECTADY, NY (US)

(21) Appl. No.: 12/463,822

(22) Filed: May 11, 2009

(51) Int. Cl. A61N 7/00; A61B 8/13

(52) U.S. Cl. 600/439

(57) ABSTRACT

An ultrasound imaging and therapy system that includes an ultrasound probe and an ultrasound diagnostic module to control the probe to obtain diagnostic ultrasound signals from a region of interest (ROI). The ROI includes adipose tissue and non-adipose tissue. The diagnostic module analyzes the diagnostic ultrasound signals and automatically differentiates adipose tissue from non-adipose tissue. The system also includes an ultrasound therapy module to control the probe to deliver, during a therapy session, a therapy at a treatment location based on a therapy parameter to the adipose tissue differentiated by the ultrasound diagnostic module. A method for delivering therapy to a region of interest (ROI) in a patient is also provided.

### CROSS-REFERENCE TO RELATED APPLICATIONS

This application includes subject matter that is similar to the subject matter described in U.S. Patent Applications having Attorney Docket No. 235594 (555-0003US), entitled “ULTRASOUND SYSTEM AND METHOD TO DELIVER THERAPY BASED ON USER DEFINED TREATMENT SPACES,” and Attorney Docket No. 235610 (555-0005US), entitled “ULTRASOUND SYSTEM AND METHOD TO DETERMINE MECHANICAL PROPERTIES OF A TARGET REGION,” both of which are filed contemporaneously herewith and are incorporated by reference in their entirety.

### BACKGROUND OF THE INVENTION

The subject matter herein relates generally to diagnostic imaging and therapy systems that provide diagnostic imaging and treatment of a region of interest in a patient, and more particularly, to ultrasound systems that image and treat adipose tissue.

Various body contouring systems exist today that attempt to remove or destroy fatty tissue (or adipose tissue) from a person's body. Some systems may be invasive, such as liposuction, where a device is inserted into the body and physically removes adipose tissue through suction. Other systems may be non-invasive. For example, in one non-invasive system high-intensity focused ultrasound (HIFU) signals are directed toward a region within the adipose tissue. The HIFU signals may at least partially liquefy the adipose tissue through lysing or causing cavitation or thermal damage of the cells within the adipose tissue.

However, since the ultrasound signals may have a harmful effect on the non-adipose tissue, it is important for a user of a HIFU system to know and control where treatment has been provided within the body of a patient. In one known system, a user draws an outline of a region on a surface of the body where treatment will be provided and also applies markers to the surface around or within the outline on the body of the patient. A video camera is positioned over the body and oriented to view the surface of the patient's skin where therapy is applied. The HIFU system tracks the progress of the therapy based upon the location of the outline on the body and the markers.

The HIFU system described above has certain limitations. For example, the HIFU system may only display the surface of the patient's skin and does not provide a visual representation or image of the volume of the body under the surface. Consequently, the above HIFU system does not provide control for localizing therapy to certain regions under the surface of the skin. Further, the above conventional HIFU system also does not know or determine where non-adipose tissue may be located with respect to the adipose tissue. Therefore, the HIFU system does not automatically differentiate the tissues. The HIFU system may also not confirm that therapy has been delivered to the desired regions.

Accordingly, there is a need for ultrasound imaging and therapy systems that indicate where, within a volume of the patient, therapy has been provided or will be provided. Furthermore, there is a need for systems that facilitate a user of the system in identifying a treatment space of a region of interest beneath the surface and applying treatment to the space.

### BRIEF DESCRIPTION OF THE INVENTION

In one embodiment, an ultrasound imaging and therapy system that includes an ultrasound probe and an ultrasound diagnostic module to control the probe to obtain diagnostic ultrasound signals from a region of interest (ROI). The ROI includes adipose tissue and non-adipose tissue. The diagnostic module analyzes the diagnostic ultrasound signals and automatically differentiates adipose tissue from non-adipose tissue. The system also includes an ultrasound therapy module to control the probe to deliver, during a therapy session, a therapy at a treatment location based on a therapy parameter to the adipose tissue differentiated by the ultrasound diagnostic module.

In another embodiment, a method for delivering therapy to a region of interest (ROI) in a patient is provided. The method includes obtaining diagnostic ultrasound signals from a region of interest (ROI). The ROI includes adipose tissue and non-adipose tissue. The method also includes analyzing the diagnostic ultrasound signals and automatically differentiating adipose tissue from non-adipose tissue. Also, the method includes delivering therapy, during a therapy session, at a treatment location based on a therapy parameter to the adipose tissue differentiated by the ultrasound diagnostic module.

### BRIEF DESCRIPTION OF THE DRAWINGS

FIG. 1 is a block diagram of an ultrasound system formed in accordance with an embodiment of the invention.

FIG. 2 is a block diagram of a diagnostic module in the ultrasound system of FIG. 1 formed in accordance with an embodiment of the invention.

FIG. 3 is a block diagram of a therapy module in the ultrasound system of FIG. 1 formed in accordance with an embodiment of the invention.

FIG. 4 illustrates a window presented on a display of FIG. 1 that displays a treatment space of a region of interest.

FIG. 5 illustrates windows that may be presented on the display of FIG. 1.

FIG. 6 illustrates another window presented on a display of FIG. 1 that displays a treatment space of a region of interest.

FIG. 7 shows the window in FIG. 6 as the ultrasound system delivers therapy to the treatment space.

FIG. 8 is an image of a C-plane view of a region of interest.

FIG. 9 illustrates an ultrasound system in accordance with one embodiment that includes a tracking system and a registering system.

FIG. 10 illustrates transducer arrays that may be used with a probe in accordance with various embodiments.

FIG. 11 illustrates an ultrasound system in accordance with one embodiment that includes a device for removing adipose tissue from a patient during a therapy session.

FIG. 12 is a flowchart illustrating a method in accordance with one embodiment.

FIG. 13 illustrates a hand carried or pocket-sized ultrasound imaging system that may be configured to display a region of interest during a therapy session in accordance with various embodiments.

FIG. 14 illustrates a console-based ultrasound imaging system provided on a movable base that may be configured to display a region of interest during a therapy session in accordance with various embodiments.

FIG. 15 is a block diagram of exemplary manners in which embodiments of the invention may be stored, distributed, and installed on computer readable medium.

### DETAILED DESCRIPTION OF THE INVENTION

Exemplary embodiments that are described in detail below include ultrasound systems and methods for imaging and treating a region of interest (ROI). The ROI may include adipose tissue and/or non-adipose tissue, such as muscle tissue, bone, tissue of organs, and blood vessels. The system may display the ROI so that an operator or user of the system can distinguish the adipose tissue and the non-adipose tissue and/or the system may automatically differentiate the adipose tissue and the non-adipose tissue prior to treating. Treatment of the ROI may include providing high-intensity focused ultrasound (HIFU) signals to treatment locations within the ROI. For example, HIFU signals may be directed to treatment locations within the adipose tissue to at least partially liquefy the adipose tissue. Liquefication may occur through cell lysis, cavitation, and/or thermal damage in the adipose tissue.

The following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

It should be noted that although the various embodiments may be described in connection with an ultrasound system, the methods and systems described herein are not limited to ultrasound imaging. In particular, the various embodiments may be implemented in connection with different types of medical imaging, including, for example, magnetic resonance imaging (MRI) and computed-tomography (CT) imaging. Further, the various embodiments may be implemented in other non-medical imaging systems, for example, non-destructive testing systems, such as airport screening systems.

A technical effect of the various embodiments of the systems and methods described herein include generating an image of a ROI and accepting user inputs to designate a treatment space within the ROI that corresponds to adipose tissue. Another technical effect may include providing therapy to treatment locations and automatically moving the treatment location between multiple points (or treatment sites) within the treatment. In some embodiments, another technical effect includes analyzing the diagnostic ultrasound signals and automatically differentiating adipose tissue from non-adipose tissue. Other technical effects may be provided by the embodiments described herein.

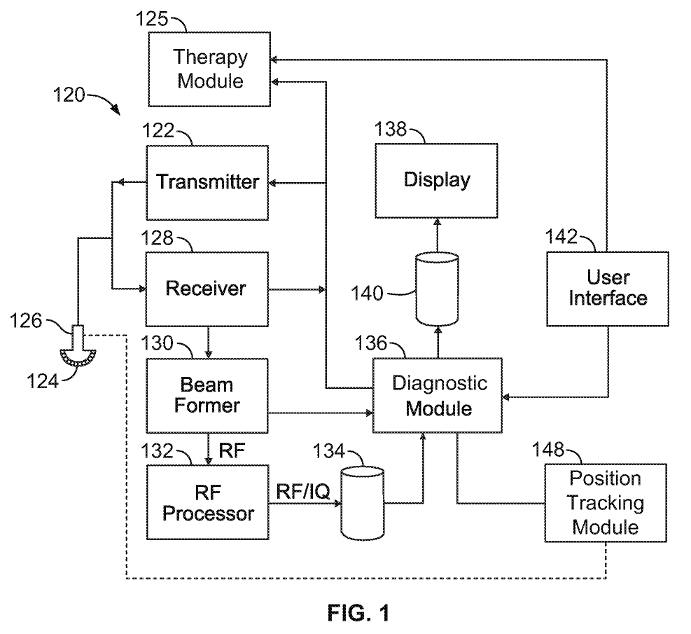

FIG. 1 is a block diagram of an exemplary ultrasound imaging and therapy system 120 in which the various embodiments can display and provide therapy to a ROI as described in more detail below. The ultrasound system 120 includes a transmitter 122 that drives an array of transducer elements 124 (e.g., piezoelectric crystals) within a probe 126 to emit pulsed ultrasonic signals into a body or volume. The pulsed ultrasonic signals may be for imaging and for therapy of the ROI. For example, the probe 126 may deliver low energy pulses during imaging and high energy pulses during therapy. A variety of geometries may be used and the array of transducer elements 124 may be provided as part of, for example, different types of ultrasound probes.

The imaging signals are back-scattered from structures in the body, for example, adipose tissue, muscular tissue, connective tissue, blood cells, veins or objects within the body (e.g., a catheter or needle) to produce echoes that return to the elements 124. The echoes are received by a receiver 128. The received echoes are provided to a beamformer 130 that performs beamforming and outputs an RF signal. The RF signal is then provided to an RF processor 132 that processes the RF signal. Alternatively, the RF processor 132 may include a complex demodulator (not shown) that demodulates the RF signal to form IQ data pairs representative of the echo signals. The RF or IQ signal data may then be provided directly to a memory 134 for storage (e.g., temporary storage). Optionally, the output of the beamformer 130 may be passed directly to the diagnostic module 136.

The ultrasound system 120 also includes a processor or diagnostic module 136 to process the acquired ultrasound information (e.g., RF signal data or IQ data pairs) and prepare frames of ultrasound information for display on a display 138. The diagnostic module 136 is adapted to perform one or more processing operations according to a plurality of selectable ultrasound modalities on the acquired ultrasound information. Acquired ultrasound information may be processed in real-time during a scanning or therapy session as the echo signals are received. Additionally or alternatively, the ultrasound information may be stored temporarily in the memory 134 during a scanning session and processed in less than real-time in a live or off-line operation. An image memory 140 is included for storing processed frames of acquired ultrasound information that are not scheduled to be displayed immediately. The image memory 140 may comprise any known data storage medium, for example, a permanent storage medium, removable storage medium, etc.

The diagnostic module 136 is connected to a user interface 142 that controls operation of the diagnostic module 136 as explained below in more detail and is configured to receive inputs from a user. The display 138 includes one or more monitors that present patient information, including diagnostic and therapeutic ultrasound images to the user for review, diagnosis, analysis, and treatment. The display 138 may automatically display, for example, a 2D, 3D, or 4D ultrasound data set stored in the memory 134 or 140 or currently being acquired, which data set is also displayed with a graphical representation (e.g., an outline of a treatment space or a marker within the treatment space). One or both of the memory 134 and the memory 140 may store 3D data sets of the ultrasound data, where such 3D data sets are accessed to present 2D and 3D images. For example, a 3D ultrasound data set may be mapped into the corresponding memory 134 or 140, as well as one or more reference planes. The processing of the data, including the data sets, may be based in part on user inputs, for example, user selections received at the user interface 142.

The diagnostic module 136 is configured to analyze ultrasound signals and automatically differentiate adipose tissue from non-adipose tissue (e.g., muscle tissue., bone, connective tissue, organs). The diagnostic module 136 may also be configured to receive user imaging commands for outlining or otherwise providing an overlay that indicates a treatment space within the ROI. The diagnostic module 136 may also receive user therapy commands (e.g., through the user interface 142) regarding how to apply therapy to treatment locations within the ROI. The therapy commands may relate to therapy parameters and the like.

The diagnostic module 136 communicates with a therapy module 125 that is configured to control the probe 126 during a therapy session. A “therapy session,” as used herein, is a period of time in which a patient receives therapy. For example, a therapy session may include a single application of ultrasounds signals to liquefy adipose tissue at a single treatment location or within a single treatment space within the body. A therapy session may also include an extended period of time in which a patient receives multiple applications of ultrasound signals within a treatment space of one region of the body or within multiple regions of the body. A therapy session may also include one visit by a patient to an operator of the system 120.

The diagnostic module 136 is configured to control the probe 126 to obtain diagnostic ultrasound signals from the ROI, and the therapy module 125 is configured to deliver a therapy to the treatment locations based on one or more therapy parameters. The therapy module 125 may automatically move the treatment location between multiple points based on user inputs.

In operation, the system 120 acquires data, for example, volumetric data sets by various techniques (e.g., 3D scanning, real-time 3D imaging, volume scanning, 2D scanning with transducers having positioning sensors, freehand scanning using a voxel correlation technique, scanning using 2D or matrix array transducers, etc.). The data may be acquired by moving the probe 126, such as along a linear or curvilinear path, while scanning the ROI. At each linear or arcuate position, the probe 126 obtains scan planes that are stored in the memory 134. The probe 126 also may be mechanically moveable within the ultrasound probe.

Optionally, the system 120 may include a position tracking module 148 that tracks a position of the probe 126 and communicates the position to the diagnostic module 136. A position of the probe 126 may be tracked relative to a reference point on or near the patient, a marker, and the like. As will be described in greater detail below, the position of the probe 126 may be used to indicate, to the user, regions of the patient that have already been treated, are being treated, or have yet to be treated.

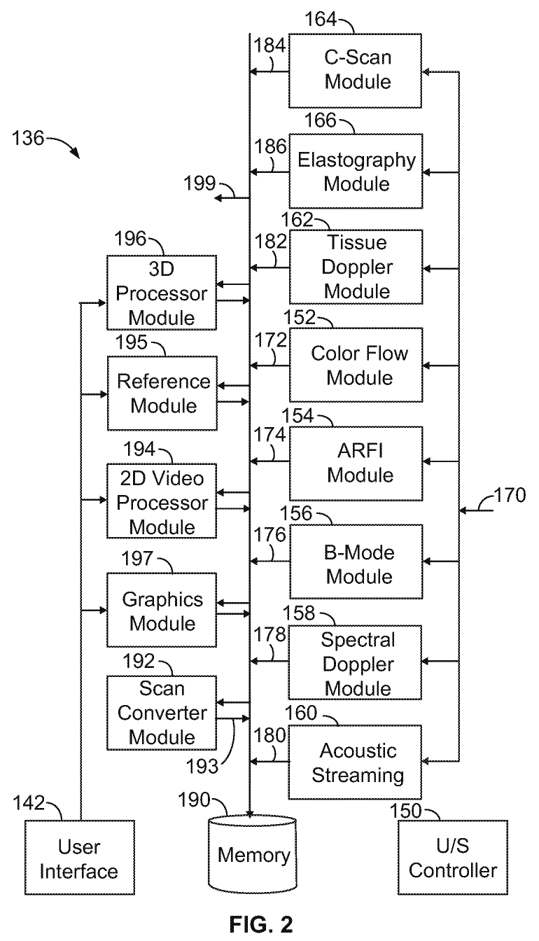

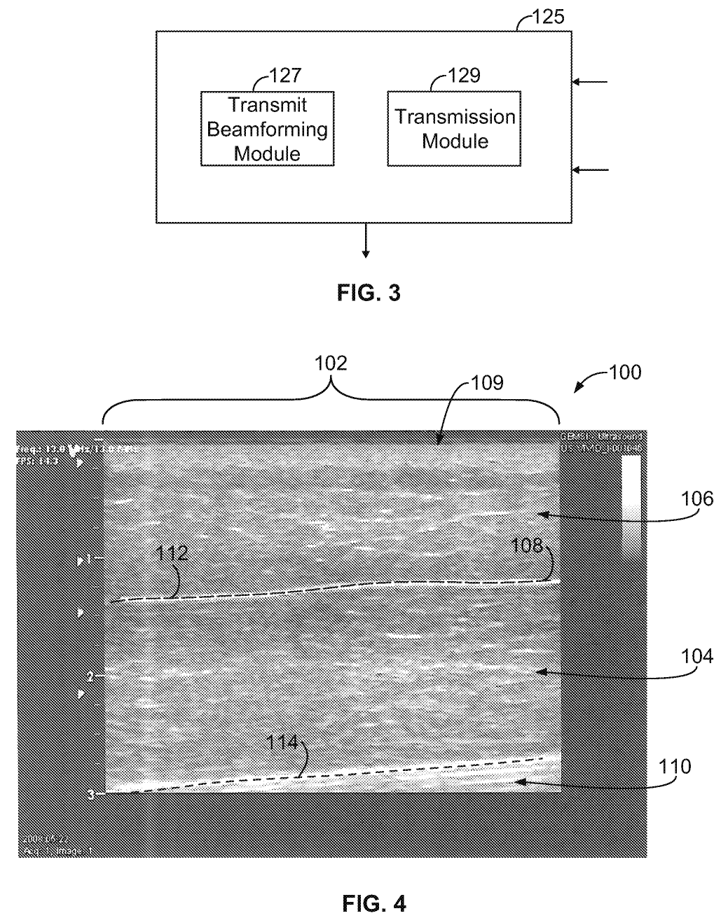

FIG. 2 is an exemplary block diagram of the diagnostic module 136, and FIG. 3 is an exemplary block diagram of the therapy module 125. The therapy and diagnostic modules 125 (FIG. 3) and 136 (FIG. 2) are illustrated conceptually as a collection of modules, but may be implemented utilizing any combination of dedicated hardware boards, DSPs, processors, etc. Alternatively, the modules of FIGS. 2 and 3 may be implemented utilizing an off-the-shelf PC with a single processor or multiple processors, with the functional operations distributed between the processors. As a further option, the modules of FIGS. 2 and 3 may be implemented utilizing a hybrid configuration in which certain modular functions are performed utilizing dedicated hardware, while the remaining modular functions are performed utilizing an off-the-shelf PC and the like. The modules also may be implemented as software modules within a processing unit. Furthermore, the diagnostic module 136 may include the therapy module 125, or the therapy module 125 may include the diagnostic module 136.

The operations of the modules illustrated in FIGS. 2 and 3 may be controlled by a local ultrasound controller 150 or by the diagnostic module 136. The modules 152-166 perform mid-processor operations. The diagnostic module 136 may receive ultrasound data 170 in one of several forms. In the embodiment of FIG. 2, the received ultrasound data 170 constitutes IQ data pairs representing the real and imaginary components associated with each data sample. The IQ data pairs are provided to one or more modules, for example, a color-flow module 152, an acoustic radiation force imaging (ARFI) module 154, a B-mode module 156, a spectral Doppler module 158, an acoustic streaming module 160, a tissue Doppler module 162, a C-scan module 164, and an elastography module 166. Other modules may be included, such as an M-mode module, power Doppler module., harmonic tissue strain imaging, among others. However, embodiments described herein are not limited to processing IQ data pairs. For example, processing may be done with RF data and/or using other methods. Furthermore, data may be processed through multiple modules.

Each of modules 152-166 are configured to process the IQ data pairs in a corresponding manner to generate, respectively, color-flow data 172, ARFI data 174, B-mode data 176, spectral Doppler data 178, acoustic streaming data 180, tissue Doppler data 182, C-scan data 184, elastography data 186, among others, all of which may be stored in a memory 190 (or memory 134 or image memory 140 shown in FIG. 1) temporarily before subsequent processing. The data 172-186 may be stored, for example, as sets of vector data values, where each set defines an individual ultrasound image frame. The vector data values are generally organized based on the polar coordinate system.

A scan converter module 192 accesses and obtains from the memory 190 the vector data values associated with an image frame and converts the set of vector data values to Cartesian coordinates to generate an ultrasound image frame 193 formatted for display. The ultrasound image frames 193 generated by the scan converter module 192 may be provided back to the memory 190 for subsequent processing or may be provided to the memory 134 (FIG. 1) or the image memory 140 (FIG. 1). Once the scan converter module 192 generates the ultrasound image frames 193 associated with the data, the image frames may be restored in the memory 190 or communicated over a bus 199 to a database (not shown), the memory 134, the image memory 140 and/or to other processors (not shown).

As an example, it may be desired to view different ultrasound images relating to a therapy session in real-time on the display 138 (FIG. 1). To do so, the scan converter module 192 obtains data sets for images stored in the memory 190 of that are currently being acquired. The vector data is interpolated where necessary and converted into an X, Y format for video display to produce ultrasound image frames. The scan converted ultrasound image frames are provided to a display controller (not shown) that may include a video processor that maps the video to a gray-scale mapping for video display. The gray-scale map may represent a transfer function of the raw image data to displayed gray levels. Once the video data is mapped to the gray-scale values, the display controller controls the display 138, which may include one or more monitors or windows of the display, to display the image frame. The image displayed in the display 138 is produced from an image frame of data in which each datum indicates the intensity or brightness of a respective pixel in the display.

Referring again to FIG. 2, a 2D video processor module 194 may be used to combine one or more of the frames generated from the different types of ultrasound information. For example, the 2D video processor module 194 may combine different image frames by mapping one type of data to a gray map and mapping the other type of data to a color map for video display. In the final displayed image, the color pixel data is superimposed on the gray scale pixel data to form a single multi-mode image frame that is again re-stored in the memory 190 or communicated over the bus 199. Successive frames of images may be stored as a cine loop (4D images) in the memory 190 or memory 140 (FIG. 1). The cine loop represents a first in, first out circular image buffer to capture image data that is displayed in real-time to the user. The user may freeze the cine loop by entering a freeze command at the user interface 142. The user interface 142 may include, for example, a keyboard and mouse and all other input controls associated with inputting information into the ultrasound system 120 (FIG. 1). In one embodiment, the user interface 142 includes the display 138 that may be touch-sensitive or configured to interact with a stylus. The user interface 142 may also receive user inputs through voice-recognition or activation.

A 3D processor module 196 is also controlled by the user interface 142 and accesses the memory 190 to obtain spatially consecutive groups of ultrasound image frames and to generate three-dimensional image representations thereof, such as through volume rendering or surface rendering algorithms as are known. The three-dimensional images may be generated utilizing various imaging techniques, such as ray-casting, maximum intensity pixel projection and the like.

A graphic module 197 may also be controlled by the system 120 and may access the memory 190 to obtain groups of ultrasound image frames that have been stored or that are currently being acquired. The graphic module 197 may generate images that include the images of the ROI and a graphical representation positioned (e.g., overlaid) onto the images of the ROI. The graphical representation may represent an outline of a treatment space, the focal point or region of the therapy beam, a path taken by the focal region within the treatment space, a probe used during the session, and the like. Graphical representations may also be used to indicate the progress of the therapy session. The graphical representations may be generated using a saved graphical image or drawing (e.g., computer graphic generated drawing), or the graphical representation may be directly drawn by the user onto the image using a pointing device, e.g., an electronic stylus or mouse, or another interface device.

Also shown, a reference module 195 may be used to identify a reference point on or near the patient during the therapy session. For example, a reference point may be an anatomical element or structure of the body that is determined by the system 120 or by the user. The reference point may also be an element or marker positioned on the surface of the body of the patient. As will be described in greater detail below, the reference module 195 may use the imaging data to determine a relation of the treatment space with respect to a reference point.

Referring to FIG. 3, the therapy module 125 may be coupled to the diagnostic module 136 (FIG. 2) and the user interface 142 (FIG. 1) and include a transmit beamforming module 127 and a transmission module 129. The therapy module 125 and the diagnostic module 136 may also be a common module or processor. The transmit beamforming module 127 is configured to control the location and movement of a focal point or region generated by the transducer elements 124. For example, the transmit beamforming module 127 may control electronic or mechanical steering of the probe to move the focal region of a therapy beam within the treatment spaces or between different treatment spaces. The transmission module 129 is configured to drive the transducer elements 124 (or only a portion or subset of the transducer elements 124) in delivering energy pulses to the ROI for imaging and therapy.

FIG. 4 illustrates a window 100 that may be presented on the display 138 (FIG. 1). The display 138 communicates with the diagnostic module 136 (FIG. 1) to display an image 102 of the ROI of the patient within the window 100. As shown in the image 102, the ROI may include adipose layers or tissues 104 and 106 and non-adipose layers or tissues, such as dermis layer 109, connective tissue 108, and muscle tissue 110. The image 102 of the ROI may also include other anatomical structures such as bone, organs, cartilage, and others. As will be discussed in greater detail, the system 120 may be able to automatically identify or differentiate between the layers. After differentiating the adipose tissue 104 from other tissues, the system 120 may deliver therapy pulses (e.g., HIFU) to at least one of the adipose tissue 104 and 106. Alternatively, the user of the system 120 may be able to recognize or identify through the image 102 the different layers of tissue. The user may then enter user inputs for designating spaces within at least one of the adipose tissue 104 and 106 for receiving therapy.

In order to automatically differentiate the adipose tissue 104, 106 from the non-adipose tissues and, more specifically, the dermis tissue 109, connective tissue 108, and muscle tissue 110, the diagnostic module 136 may analyze ultrasound signals received from the ROI. For instance, the diagnostic module 136 may use data obtained through one or more processing methods (e.g., B-mode, elastography, color-flow). In some embodiments, the diagnostic module 136 automatically differentiates the adipose tissue 104 from the non-adipose tissues by at least identifying barriers or boundaries 112 and 114 (indicated as hashed lines) between the adipose tissues 104 and 106 and/or an adjacent non-adipose tissue, such as the dermis tissue 109 and the muscle tissue 110. For example, the diagnostic module 136 may identify the connective tissue 108 that extends between the adipose tissues 104 and 106. The system 120 (FIG. 1) may use stored information regarding general human anatomy at the ROI to identify a location of the adipose tissue 104 in relation to the boundary 112 (i.e., the connective tissue 108 shown in FIG. 4). More specifically, the system 120 may identify the adipose tissue 104 as the tissue or layer extending directly beneath the connective tissue 108. Optionally, the system 120 may indicate to the operator the different layers and tissues within the ROI by illustrating graphical representations of the barriers or boundaries 112 and 114, such as overlaying hashed lines onto the image 102 as shown in FIG. 4.

The system 120 may automatically differentiate tissues within the ROI using other methods separately or in conjunction with identifying barriers or boundaries. As another example, the diagnostic module 136 may automatically differentiate the adipose tissue from other tissues by at least directly measuring a plurality of points within the ROI for an adipose characteristic. The adipose characteristic may include a measure of the tissue mechanical properties at one or more points, such as a tissue stiffness, a shear wave velocity between points, a longitudinal wave velocity between points, and a density of the tissue at one or more points. The adipose characteristic may also relate to thermal properties, including thermal conductivity and specific heat. Another adipose characteristic may be cavitation inducibility provided that non-adipose tissue would not be damaged under the conditions that produced cavitation in adipose tissue.

In addition to identifying the adipose tissue 104 by measuring for an adipose characteristic, the diagnostic module 136 may identify other tissues by measuring for non-adipose characteristics. For example, the diagnostic module 136 may identify the connective tissue 108 and the muscle tissue 110 by measuring tissue mechanical properties at one or more points within the ROI, such as a tissue stiffness, a shear wave velocity between points, a longitudinal wave velocity between points, and a density of the tissue at one or more points. The non-adipose characteristic may also relate to thermal properties.

Furthermore, techniques using one or more modules 152-166 (FIG. 2) and/or the methods described above may be used to examine patterns in the image that are characteristic of different types of tissues (e.g., adipose tissue, muscle tissue, connective tissue, bone tissue). For instance, a layered appearance that is characteristic of muscle tissue may be determined, or a cellular structure of adipose tissue caused by connective tissue boundaries could be determined.

As a more specific example, in some embodiments, the diagnostic module 136 may analyze images obtained through B-mode processing. A B-mode image is an image that may be gray-scale or colored showing a cross-section of the ROI along a scanning plane. The B-mode image relates to acoustic backscatter energy from different tissues within the ROI. FIG. 4 illustrates a B-mode gray-scale image of the ROI. As shown, different tissues may have different brightness levels. The diagnostic module 120 may measure a brightness level of a plurality of points within the image 102 to differentiate the adipose tissue 104 relative to other tissues. Furthermore, the diagnostic module 136 may use stored information, such as the expected anatomical structures for a particular ROI, in addition to the measured brightness levels to determine the adipose tissue 104. As one example, if the ROI is of the abdomen, the diagnostic module 136 may expect the image 102 to include the dermis tissue 106 and the adipose tissue 104 separated by the connective tissue 108.

Also, the system 120 may use elastography processing methods alone or in addition to other processing methods to differentiate the adipose tissue 104. In one type of ultrasound elastography, a compression is applied to the ROI (e.g., along the skin surface) and strain images of the tissues in response to the compression may be obtained. For example, speckle tracking techniques may use data obtained before and after the applied compression to calculate the relative motion of different types of tissues due to their different mechanical properties (e.g., tissue strain or stiffness). As such, the images obtained through elastography, also called elastograms, relate to elastic properties of the tissues. Moreover, other techniques may be used to examine the mechanical properties of different tissues. For example, other elastography techniques, such as inducing and measuring the shear waves or vibro-acoustography, may be used to differentiate adipose tissue from other tissue types.

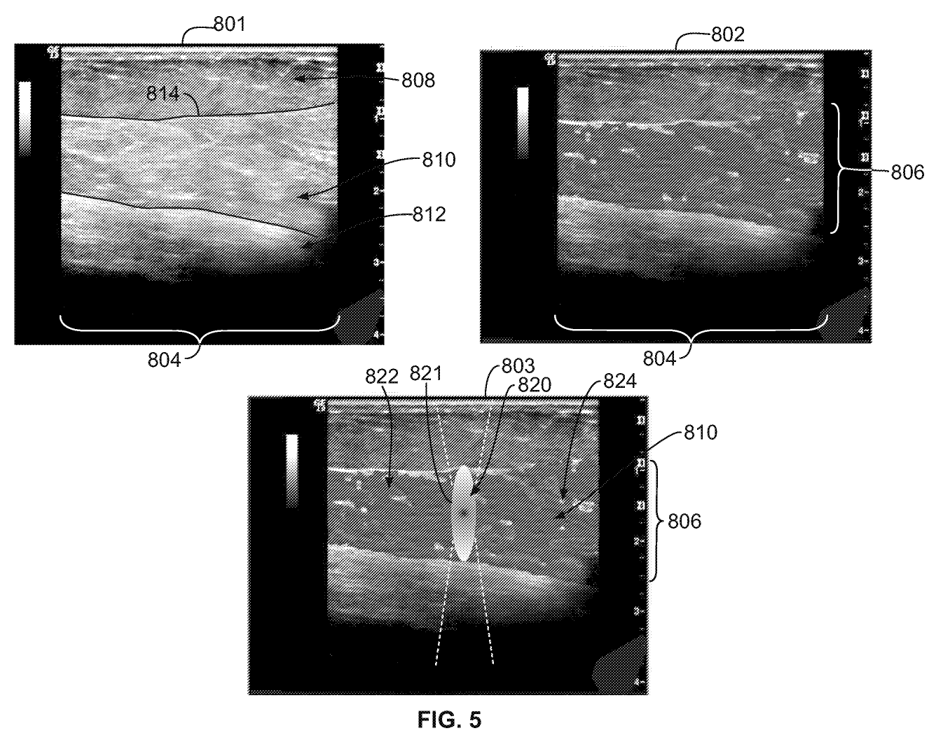

FIG. 5 shows three windows 801-803 that may be shown together or separately on the display 138 (FIG. 1). More specifically, FIG. 5 illustrates an embodiment where the diagnostic module 136 (FIG. 1) uses data obtained through a B-mode processing method and data obtained through an elastographic processing method. The window 801 includes a B-mode image 804 of a ROI. As discussed above, the diagnostic module 136 may use data from the B-mode processing method and/or the elastographic processing method to automatically differentiate adipose tissue 808 and 810 from non-adipose tissues 812 and 814. The window 802 illustrates the B-mode image 804 and an elastographic image 806 (also referred to as an elastogram) that overlay one another or are integrated with each other. (Although not shown in FIG. 5, the elastogram 806 may be colored (e.g., red) and overlay the black-and-white or gray-scale B-mode image 804 so as to provide a reddish tinge or shading.) The window 802 may represent the ROI before applying therapy.

The window 803 is an example of a window that an operator may see on the display 138 (FIG. 1) during a therapy session. As shown, a treatment location 820 may be indicated by a graphical representation 821, such as an elongated oval. The window 803 shows a treated space 822 to the left of the treatment location 820 that has already received therapy and an untreated space 824 to the right of the treatment location 820 that has not received therapy. When therapy is applied and HIFU signals are directed to the treatment location 820, the adipose tissue 810 may be at least partially liquefied at or proximate to the treatment location 820. Accordingly, mechanical properties of the adipose tissue 810 corresponding to the treated space 822 will be different than mechanical properties of the adipose tissue 810 corresponding to the untreated space 824. During treatment of the adipose tissue 810, the elastogram 806 may be updated. Spaces having different mechanical properties may be visually different in the elastogram 806 and, consequently, the window 803. For example, a portion of the elastogram 806 corresponding to the treated space 822 may have a greenish color or shading. A portion of the elastogram 806 corresponding to the untreated space 824 may have a reddish color or shading. Accordingly, the window 803 may indicate to the operator the spaces or areas of the ROI in the patient that have been treated, that are being treated, or that have not been treated.

Furthermore, the elastogram 806 is not limited to showing two colors where one is for the treated space and the other is for the untreated space. The elastogram 806 may show a spectrum of colors that are indicative of the mechanical properties of the adipose tissue 810. For example, if the therapy applied to the treatment location 820 does not liquefy the adipose tissue 810 at the treatment location 820, the color (or some other visual indication) in the elastogram 806 may indicate to the operator that the treatment only partially liquefied the adipose tissue 810.

In some embodiments, after the initial scan to obtain diagnostic ultrasound signals has been performed and the adipose tissue has been identified, the diagnostic module 136 may automatically set a therapy parameter before therapy is applied to the ROI. For example, the diagnostic module 136 may identify a thickness of a layer of the adipose tissue. Based on the thickness of the adipose tissue and/or other factors, the diagnostic module 136 may automatically set a focal region depth, a focal region size, an ablation time for each point within the ROI that receives therapy, an energy level of the therapy signals, and a rate of focal region movement within the ROI during the therapy session. Other parameters may be automatically set, including a peak negative pressure, a pulse repetition rate, a duty cycle, a dwell time, and pulse sequences. Also, if the thickness, depth, or density of the layer of the adipose tissue(s) decreases or increases as the adipose tissue extends laterally across the ROI (e.g., across the image 102 (FIG. 4)), the therapy parameters may change in accordance with the thickness, density, or depth of the layer as the therapy is applied. For example, if the thickness is decreasing, then the focal region size may also decrease or the energy level of the therapy signals may decrease as the therapy moves along the adipose tissue 104.

After or while therapy is applied within the ROI, the diagnostic module 136 may analyze ultrasound signals from a second supplemental scan to confirm treatment of the ROI. For example, the diagnostic module 136 may obtain images from the second scan that include data processed through elastography methods. After or while the adipose tissue receives therapy, images obtained through elastography methods may indicate a change in tissue stiffness at a location that has received or is receiving therapy. More specifically, an initial image may have a color, brightness, or density that indicates a tissue stiffness of the adipose tissue before treatment. In the second supplemental scan, the color, brightness, or density of the adipose tissue may change where the adipose tissue received therapy. The images from the initial and supplemental scans may be used by the system 120 to confirm that treatment was received within the adipose tissue. For example, the images from the initial and supplemental scans may be superimposed with each other or shown side-by-side on the display 138. The operator may confirm that treatment was received within the adipose tissue by comparison of the images.

In addition or alternatively, the diagnostic module 136 may use other processing methods to facilitate determining whether a characteristic of the adipose tissue or another tissue has changed due to therapy. Also, after evaluating the ROI in the second supplemental scan, the therapy parameters may be adjusted if therapy is to be applied again to the ROI or if the focal region moves to another location within the ROI.

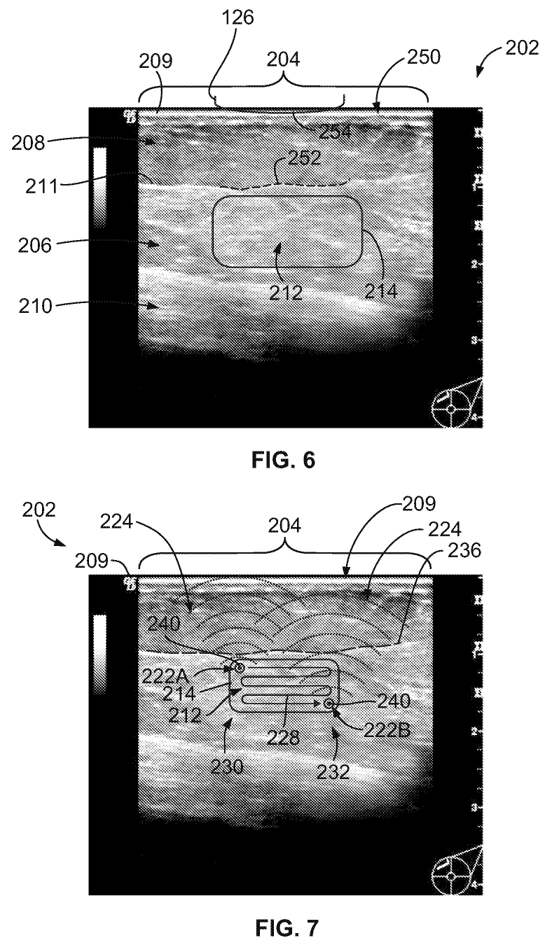

FIGS. 6 and 7 also illustrate features of the system 120 that may be used by an operator during a therapy session to facilitate delivering therapy to the ROI. FIG. 6 illustrates a window 202 that may be presented on the display 138 (FIG. 1). As shown in an image 204, the ROI includes adipose tissue 206 and 208 and non-adipose tissues, such as dermis tissue 209, muscle tissue 210, and connective tissue 211. In some embodiments, the system 120 automatically designates a treatment space 212 or the user interface 142 (FIG. 1) accepts user inputs for designating the treatment space 212 within the ROI. The treatment space 212 represents a space that will be treated during a therapy session and is generally located within an adipose tissue, such as the adipose tissue 206. The designated treatment space 212 may correspond to a portion of the adipose tissue 206 within the image 204 or the treatment space 212 may correspond to all of the adipose tissue 206 within the ROI. By way of example, the treatment space 212 may be located and shaped so that the treatment space 212 is a distance away from the non-adipose tissues 208 and 210. As such, a probability of therapy being inadvertently applied to spaces outside of the treatment space 212, such as the non-adipose tissues 211 and 210, may be decreased.

The display 138 may indicate to the user or another viewer (e.g., the patient or a person in a remote location) the treatment space 212 designated by the user inputs. A graphical representation, such as an outline 214, may be overlaid upon the image 204. The outline 214 designates boundaries of the treatment space 212 to indicate to a viewer where the therapy will be applied. The outline 214 may be determined by parameters entered by the user. For example, the user may select pre-programmed outlines 214 or may enter coordinates or dimensions for the treatment space 212 to form the outline 214. The outline 214 may indicate an enclosed region within the treatment space 212. The outline 214 may have various shapes including a rounded rectangular shape (as shown), a parallelogram shape, another geometric shape, and the like, or a shape determined by the system 120.

The user may also enter a drawing notation to indicate where the outline 214 should be located. The drawing notation may be entered through a keyboard, a mouse, or another pointing device. As an example, the user may use a stylus pen and directly contact a touch-sensitive screen of the display 138 or a pad that is communicatively coupled to the user interface 142 to draw the drawing notation onto the image 204. As another example, the user interface 142 may recognize touches from a finger to the screen of the display 138. Furthermore, the user interface 142 may have a voice-activation module that receives voice commands from the user for entering user inputs including the drawing notation.

The outline 214 may be positioned with respect to reference points 250, 252, and 254. The reference module 195 (FIG. 2) may be configured to automatically identify the reference points 250, 252, or 254 on the patient or receive user inputs that identify the reference points. For instance, the reference point 250 may be a surface of the patient's skin or the dermis layer 209, the reference point 252 may be a particular point of or a portion of a boundary between the adipose tissues 206 and 208, and the reference point 254 may be a point along a surface of the probe 126. (For illustrative purposes, the reference point 254 and the probe 126 are shown in FIG. 6. However, the reference point 254 and the probe 126 may or may not be shown to the viewer in the image 204.) Reference points may also be other points within the ROI, such as bone, other artifacts, or a reference element such as a metallic sticker placed on a patient's skin.

After identifying a reference point, the reference module 195 may determine a relation of the treatment space 212 with respect to the identified reference point using ultrasound signal processing methods (e.g., speckle tracking). The reference module 195 may position the outline 214 of the treatment space 212 on the image 204 based on the relation of the treatment space 212 with respect to the identified reference point. As a more specific example, the reference module 195 may establish a positional relation between the adipose tissue 206 and the reference point 254 that represents a surface of the probe 126. Based on the positional relation, the reference module 195 may adjust a position of the treatment space 212 on the image 204. In other words, as the probe 126 moves along the surface of the skin or is pressed into the patient, the outline 214 on the image 204 may also move.

Furthermore, the reference module 195 may use data gathered or determined by the diagnostic module 136 (FIG. 1) to determine where to place the treatment space 212 within the ROI. More specifically, the reference module 195 may use information or data regarding the tissue characterization discussed above. For example, the reference module 195 may use the plurality of points within the ROI that were analyzed for adipose or non-adipose characteristics. Using this data, the reference module 195 may automatically locate or determine the position of the treatment space 212 within the ROI.

In some embodiments, the diagnostic module 136 may be configured to acquire the diagnostic ultrasound signals at different frame rates. A frame rate is the number of frames or images taken per second. More specifically, the diagnostic module 136 may be configured to acquire diagnostic ultrasound signals associated with different imaging spaces within the ROI at different frame rates. For example, signals from the treatment space 212 may be acquired at one frame rate while signals from other spaces outside of the treatment space 212 may be acquired at another frame rate. In one embodiment, the diagnostic module 136 is configured to acquire diagnostic ultrasound signals at a first rate in an imaging space that includes the treatment space 212 and at a slower second rate in an imaging space that excludes the treatment space 212. Alternatively, the first rate may be slower than the second rate.

FIG. 7 shows the window 202 as the system 120 (FIG. 1) delivers therapy to the treatment space 212. When therapy is applied, ultrasonic therapy signals (e.g., HIFU) from the probe 126 (FIG. 1) are directed toward a treatment location 222 (indicated as dots 222A and 222B in FIG. 7) within the treatment space 212. A treatment location 222 includes a region where a therapy beam 224 formed by ultrasound signals from the transducer elements 124 is focused (i.e., the treatment location 222 includes a focal region of the transducer elements 124) within a body of a patient. The therapy beam 224 is shaped and directed by a selected configuration and operation of the transducer elements 124. As such, the focal region of the therapy beam 224 and, consequently, the treatment location 222 may vary in size and shape within a single therapy session. When the adipose tissue 206 is treated, the therapy beam 224 that is delivered to the treatment location 222 at least partially liquefies (e.g., lyses, causes cavitation and/or thermal damage) the adipose tissue 206 within the focal region. Adipose tissue within a space that immediately surrounds the focal region may also be affected.

The therapy module 125 (FIG. 1) is configured to move the treatment location 222 throughout the treatment space 212 between multiple points or treatment sites. As used herein, “moving the treatment location between multiple points” includes moving the treatment location 222 along a therapy path 228 between a first point and an end point and also includes moving the treatment location 222 to separate and distinct points within the treatment space 212 that may or may not be adjacent to one another along a path. The therapy path 228 may be formed by separate points where therapy is applied. For example, therapy may first be applied to a first point (indicated as the treatment location 222A). After therapy has been applied to the first point, the focal region may be readjusted onto a second point along the therapy path 228 that is separate and remotely spaced from the first point. Therapy may then be applied to the second point. The process may continue along the therapy path 228 until the therapy session is concluded at an end point (indicated as the treatment location 222B). In other embodiments, the therapy may be continuously applied as the focal region is moved along the therapy path 228 in a sweeping manner. For example, therapy may be continuously applied as the treatment location 222 is moved between the first point and the end point in FIG. 7.

The therapy path 228 may have various shapes and may be pre-programmed or, alternatively, drawn by the user. As shown in FIG. 7, the therapy module 125 may direct the treatment location 222 in a sweeping manner within the treatment space 212. More specifically, the treatment location 222 may move from a first lateral location 230 proximate one side of the image 204 or outline 214 to a second lateral location that 232 is proximate an opposing side of the image 204 or the outline 214. The treatment location 222 may maintain a predetermined depth within the adipose tissue 206 as the treatment location 222 moves between the first and second lateral locations 230 and 232. In some embodiments, after the treatment location 222 is moved from the first lateral location 230 to the second lateral location 232, the depth of the treatment location 222 may be increased or decreased. As shown in FIG. 7, the treatment location 222 moves back and forth between the first and second lateral locations 230 and 232 and increases a depth of the treatment location 222 after each crossing of the treatment space 212. As such, portions of the adipose tissue 206 may avoid sustaining multiple periods of therapy. Alternatively, the depth of the treatment location 222 may gradually change as the treatment location 222 is moved in a sweeping manner. As an example, the depth of the treatment location 222 within the adipose tissue 206 may move parallel to a boundary 236 (indicated as a dashed line) between the adipose tissue 206 and the non-adipose tissue 208. The boundary 236 may or may not be shown to the viewer.

However, the therapy path 228 shown in FIG. 7 is just one example of applying therapy to multiple points within the treatment space 212. Many other therapy paths may be taken by the treatment location 222. For example, the therapy module 125 may direct the treatment location 222 in a sweeping manner between two vertical locations while changing a lateral position within the treatment space 212 after the vertical locations have been traversed. Furthermore, the treatment location 222 is not required to move between adjacent points along the therapy path 228, but may be moved to predetermined or random points within the treatment space 212 that are not adjacent to each other. For example, therapy may be applied to one corner of a treatment space 212. Subsequently, the focal region may then be readjusted to another corner and therapy may be applied.

As described above, the delivery of therapy may be based upon a therapy parameter. A therapy parameter includes any factor or value that may be determined by the system 120 or any input that may be entered by the user that affects the therapy applied to the ROI. For example, a therapy parameter may include a transducer parameter that relates to the configuration or operation of the transducer elements 124 or probe 126. Examples of a transducer parameter include a focal region depth, a focal region size, an ablation time for each point within the ROI that receives therapy, an energy level of the therapy signals, and a rate of focal region movement within the ROI during the therapy session. The transducer parameters may also include a frequency or intensity of the therapy ultrasound signals, power, peak rarefactional pressure, pulse repetition frequency and length, duty cycle, depth of field, wave form used, speed of beam movement, density of beam, cavitation priming pulse, and general pulse sequence parameters. Also, therapy parameters may include anatomical parameters, such as the location, shape, thickness, and orientation of the adipose tissue 206 and the non-adipose tissues. An anatomical parameter may also include a density of the adipose tissue 206 and the non-adipose tissues. Furthermore, therapy parameters include the type of probe 126 used during the therapy session. The age, gender, weight, ethnicity, genetics, or medical history of the patient may also be therapy parameters. After therapy has been applied to the treatment space 212, the system 120 or the operator may ad just the therapy parameters before applying therapy to the treatment space 212 again or another treatment space.

Returning to FIG. 7, in some embodiments, the display 138 may overlay another graphical representation, such as a marker 240, onto the image 204 that designates the treatment location or locations 222. The size and shape of the marker 240 may correspond to a size and shape of the focal region of the probe 126. As the therapy beam 224 moves the treatment location 222 within the treatment space 212, the display 138 may continuously update the marker 240 to cover new points within the treatment space 212 as the new points are receiving the therapy. In some embodiments, the marker 240 may only correspond to the point or points within the treatment space that are currently receiving treatment.

However, in other embodiments, the marker 240 or another graphical representation may also indicate a path within the treatment space 212 that has received therapy. For example, if the treatment location 222 is applied continuously and moved within the treatment space 212, the path may be indicated by a thick line (e.g., like a paint stroke) along the path. If the therapy is applied at separate and distinct points, a graphical representation, such as the marker 240, may be left on each point. As such, at an end of the therapy session, the image 204 may have multiple markers 240 overlaid upon the image 204 that indicate where therapy has been applied. In some embodiments, the graphical representations that indicate past therapy may remain on the image 204 indefinitely (i.e., until removed by the user or until the therapy session has concluded). In other embodiments, the graphical representations indicating past therapy may change as time progresses. Such graphical representations may indicate a time since therapy was applied, a fluidity of the tissue, a temperature, tissue stiffness, or some other characteristic of the tissue that may change with time. As an example, when therapy is first applied to a point, the graphical representation may be red to indicate that the point has recently received therapy. As time progresses, the graphical representation may fade or change into another color (e.g., blue) to indicate a predetermined amount of time has passed since therapy was applied to the point.

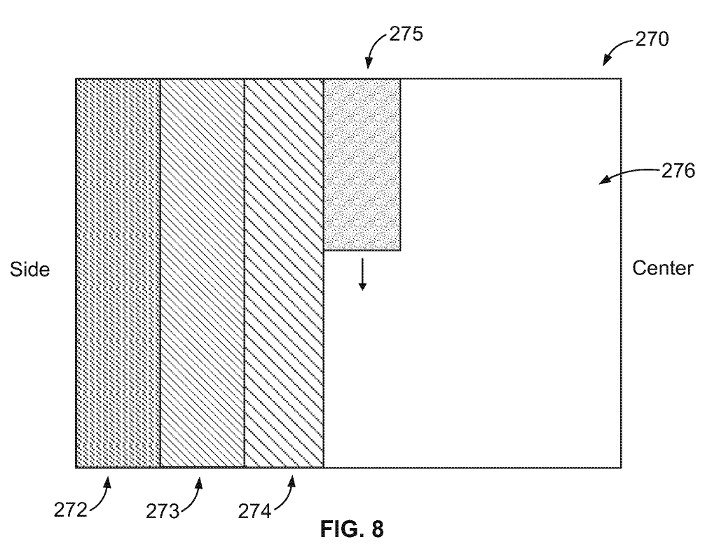

FIG. 8 is an image 270 of a C-plane view of the ROI at a predetermined depth. A C-plane view extends along a plane that does not intersect the probe 126 or the transducer elements 124. The C-plane view may be perpendicular to the view of the image 204 shown in FIGS. 6 and 7. In some embodiments, the C-plane view of the ROI is used in conjunction with the image 204. The image 270 may be provided in a window (not shown) on the display 138 concurrently with the window 202 or separately. The image 270 may also be presented on a separate display (not shown). In alternative embodiments, the image 270 is used exclusively during a therapy session (i.e., without the images 102 and 204).

The C-plane view in FIG. 8 shows an ultrasound image 270 along the C-plane at a predetermined depth. The following is with respect to one depth with the ROI. However, alter therapy has been applied to one depth of the ROI, the user of the system 120 (FIG. 1) may change depths and obtain a new C-plane view at the new depth. As shown, the image 270 illustrates sections 272-275 that indicate those spaces within the view of the image 270 that have completed treatment or a portion of treatment. Section 276 has not received any treatment. As an example, the image 270 may be of a patient's abdomen region. Section 272 may be proximate to a side of the patient and section 276 may be proximate to a center (e.g., navel) of the patient. During a therapy session, a user may apply therapy to section 272 near the patient's side.

As similarly described above, the image 270 may indicate to the user those spaces of the abdomen region that have already completed treatment. The system 120 may determine that the adipose tissue has characteristics that are different before, during, and after treatment in sections 272-275 through ultrasound signal processing methods. The characteristics may relate to, for example, a tissue stiffness or a temperature of the corresponding section. The characteristics may correlate to different or contrasting colors in the image of FIG. 8 depending upon the measured value of the characteristics. More specifically, the section 275 may have a color that indicates therapy is being currently provided or was recently provided. The section 272 may have a color that indicates therapy was applied therein a period of time ago.

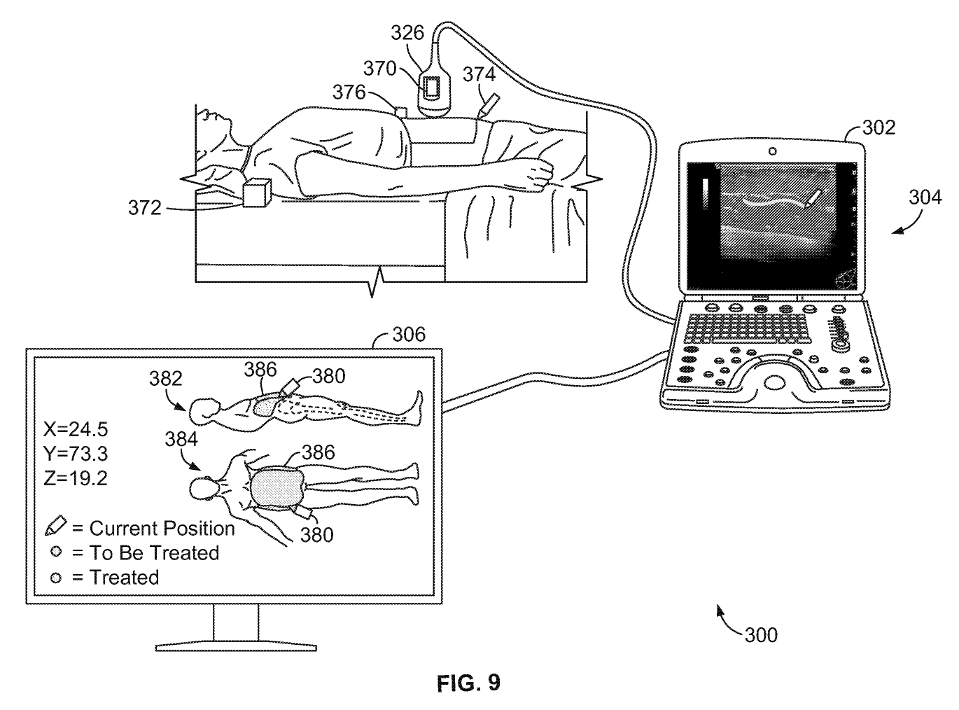

FIG. 9 illustrates an ultrasound system 300 formed in accordance with one embodiment. The system 300 may include or use similar features, components, processes, and windows as described above with respect to FIGS. 1-8. More specifically, the system 300 includes a portable computer 302 that has a primary display 304 and that is communicatively coupled to a secondary display 306. The computer 302 may also include software and internal circuitry configured to perform as described above with respect to the system 120 (FIG. 1). The system 300 includes a probe 326 that is coupled to the computer 302 and has a probe position device 370. The system also includes a reference position device 372 that may be located near the patient or may be attached to the patient. The position devices 370 and 372 may have transmitters and/or receivers that communicate with each other and/or with the computer 302. For example, the position devices 370 and 372 may communicate with a position tracking module (not shown), such as the position tracking module 148 shown in FIG. 1. The position tracking module may receive signals from the position devices 370 and/or 372. In one particular embodiment, the position device 372 has a pair of coils that creates an electromagnetic field. The position tracking module receives data (e.g., positional information) from the position devices 370 and 372 regarding a location of the probe 326. As the probe 326 applies therapy to the patient and is moved along the patient, the display 304 and/or 306 may show the movement of the probe 326 with respect to the patient.

Also shown in FIG. 9, the system 300 may be configured to register where therapy will be applied during the therapy session. The system 300 may include an electronic pen 374 and fiducial element 376 attached to the body of the patient. The fiducial element 376 is attached near the sternum of the patient in FIG. 9, but may be attached to other spaces. A user desiring to outline or delineate where therapy will be applied may use the electronic pen 374 to draw on the body of the patient. First, the electronic pen 374 may register with the fiducial element 376 so that the location of the electronic pen 374 with respect to the body of the patient is known. After registering, the electronic pen 374 moves along the surface of the body and communicates with the computer 302 a current position of the electronic pen 374. Also, the electronic pen 374 may mark the patient's body (e.g., through ink, resin, or another substance) where therapy will be applied. The computer 302 uses the data received by the electronic pen 374 and the position device 372 to indicate on the display 306 where therapy is to be applied. As shown, the display 306 may show a graphical representation 382 of a side-view of the body and a graphical representation 384 of an anterior view of the body. The computer 302 uses the information from the electronic pen 374 to outline a region 386 of the body to be treated. The region 386 may be colored green prior to treatment. In an alternative embodiment, a single element or device may perform the functions of the fiducial element 376 and the reference position device 372.

As one example, the graphical representations 382 and 384 may be digital photographs of the patient's body. When therapy is applied to the body, the computer 302 tracks the position of the probe 326. As therapy is applied, the display 306 indicates an overall progress of the therapy session. For example, the display 306 may show the user the region of the body that is currently receiving therapy, the regions of the body that have already received therapy, and the regions of the body that have yet to receive therapy. For example, the regions that have received therapy may be colored red and the regions that have not received therapy may be colored green. Also, a graphical representation 380 of the probe 326 may be shown on the display 306 to indicate a current position of the probe 326 with respect to the body.

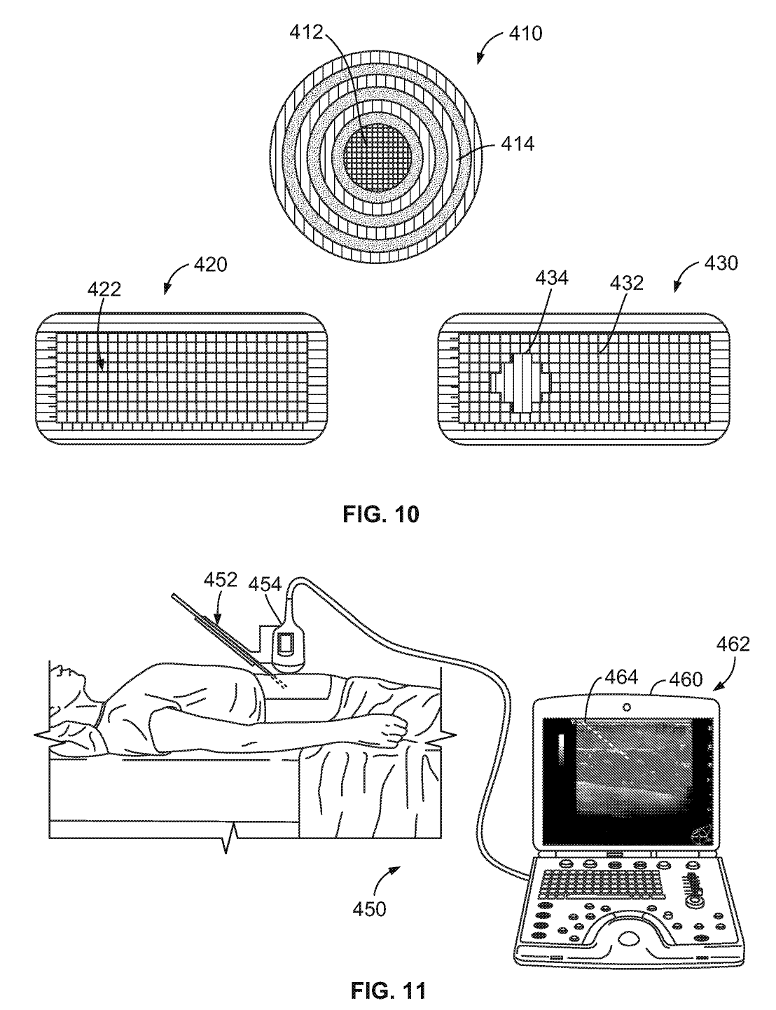

FIG. 10 illustrates transducers 410, 420, and 430 that may be used with a probe (not shown) in accordance with various embodiments. The transducers 410, 420, and 430 may include reconfigurable arrays. In some embodiments, the diagnostic module 136 (FIG. 1) and the therapy module 125 (FIG. 1) control the probe 126 (FIG. 1) to deliver low energy imaging pulses and high energy therapy pulses, respectively. The transducer 410 has an imaging array 412 and a separate therapy array 414 that surrounds the imaging array 412. The imaging array 412 and the therapy array 414 may be in a fixed relationship with respect to each other. The imaging pulses and the therapy pulses may be delivered separately or in an overlapping or interleaved manner. In some embodiments, low energy imaging pulses may also by transmitted by the therapy array 414. The reflections of these low energy imaging pulses may be received by the imaging array 412 to form an image of the focal region of the therapy array 414. Embodiments of such a dual therapy and imaging system are described in more detail in U.S. Pat. No. 5.769,790, which is incorporated by reference in the entirety.

When imaging or applying therapy to a patient, the pressure applied by the transducer to the patient's body may alter the thickness or other characteristics of the ROI, such as tissue stiffness. By combining the imaging and therapy arrays into one transducer, therapy may be applied immediately after the transducer images the ROI. As such, an accurate representation or identification of the adipose tissue may be provided immediately before the therapy is applied.

The transducer 420 includes an array 422 where the entire array may be used for both imaging and therapy. However, the transducer 430 has an array 432 of transducer elements where a therapy portion 434 of the array 432 may be activated to provide therapy. As such, the therapy module 125 may drive a subset (e.g., the therapy portion 434) of the transducer elements of the array 432 based on the user inputs designating the treatment space. Thus, the diagnostic module 136 and the therapy module 125 may deliver low energy imaging pulses and high energy therapy pulses in an interspersed manner to an at least partially overlapping array of transducer elements.

FIG. 11 illustrates an ultrasound system 450 in accordance with one embodiment that includes a device 452 for removing tissue or liquid from a patient during a therapy session. The device 452 may include a hollow tube that is inserted into the body of the patient (i.e., beneath the skin of the patient proximate to where therapy is being received). The device 452 may also include a suction device (not shown) for removing the tissue or liquid from within the ROI through the tube. The probe 454 is communicatively coupled to a computer 460 having a display 462. The display 462 may show the tube or provide a graphical representation 464 of the tube during a therapy session. The display 462 may show images of the ROI as the tissue is removed from the patient. In one embodiment, an image of the ROI before treatment may be shown side-by-side with an image of the ROI as treatment is being applied. As such, the operator of the system 450 may visually identify an amount of tissue that has been removed from the patient during the therapy session. In addition, the system 450 may incorporate other imaging, therapy, user interface, tracking, and registering features as described above with respect to system 120 and 300.

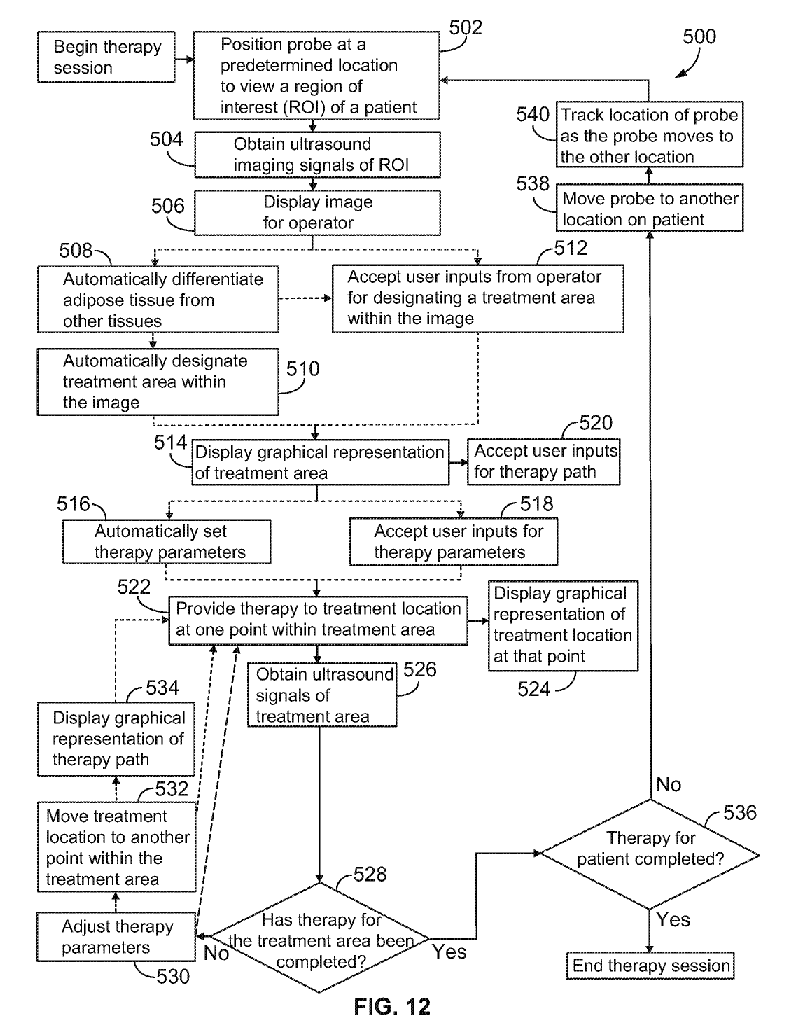

FIG. 12 is a flowchart illustrating a method 500 for delivering therapy to at least one ROI in a patient. The method 500 may be performed by a user or an operator of an imaging and therapy system. For example, the system used may be the systems 120, 300, or 450 (discussed above) or other systems described below. The therapy session may begin at 502 when the operator positions a probe at a predetermined location on the body of the patient to view an ROI. The ROI may be one of many that will be viewed during the therapy session. Ultrasound imaging signals of the ROI are obtained at 504. The signals may be processed into data via different ultrasound sub-modules, such as the modules 152-166 described above with reference to FIG. 2. In one embodiment, the signals are processed into data through at least one of B-mode and elastography processing.

An image of the ROI is generated and displayed to the operator and, optionally, patient at 506. The image may be, for example, a B-mode image in gray-scale and/or color. The image may also be a combination of images that are superimposed or arranged side-by-side with each other on a display. For example, the image may be formed from data obtained through B-mode processing and data obtained through elastography. Optionally, the system may automatically differentiate adipose tissue from other tissues at 508 and indicate to the operator the different layers of tissue within the image. The system may indicate the differentiation of tissues within the image to the user through lines, color, brightness, or other visual indications. Also, the system may automatically designate a treatment space at 510 by overlaying a graphical presentation (e.g., line) that indicates a boundary between the layers of tissue.

However, the system may also accept user inputs at 512 from the operator after a simple image (i.e., an image without graphical representations or other indications) of the ROI is displayed or after an image is displayed that automatically differentiates the tissues and indicates the different tissues to the operator. The user inputs may designate a treatment space. The system may display at 514 a graphical representation (e.g., an outline) of the designated treatment space. The system may then automatically set therapy parameters at 516 and/or the system may accept user inputs for therapy parameters at 518. Optionally, at 520, the operator may designate a therapy path within the treatment space. Therapy is then provided to a treatment location at 522 within the designated treatment space. The system may optionally, at 524, display a graphical representation (e.g., a marker) of the treatment location with the image.

After or while providing treatment to the treatment space, the system may obtain ultrasound signals of the treatment space at 526. At 528, the system determines whether treatment is complete. If treatment for the corresponding treatment space is not complete, the system may automatically adjust the therapy parameters at 530 and/or automatically move the treatment location at 532 to another point within the treatment space. The treatment location may move while providing treatment or after treatment has ended for a particular point. Furthermore, the therapy parameters may be adjusted while the treatment location is moving. Optionally, the system may display a graphical representation that indicates the path taken by the treatment location within the treatment space at 534. The system provides therapy to a new point and continues this process until the therapy for the corresponding treatment space is complete.

After therapy for the treatment space is completed, the system may determine (or ask the operator) at 536 whether therapy for the patient is complete. If therapy for the patient is complete, then the therapy session has ended. However, if the therapy session is not complete, then the system or the operator may move the probe at 538 to another location on the patient. In some embodiments, the system may also track at 540 a location of the probe as the probe moves to another location. The system may also display to the operator those regions that have already received treatment and those regions that have not received treatment.

Although the flowchart illustrates sequential steps in the method 500, embodiments herein include methods that perform fewer steps and also methods that perform the steps in different orders or may perform steps simultaneously. For example, the system may display an image of the ROI after the system has automatically differentiated the adipose tissue from other tissues. The system may also provide therapy to a treatment location within the ROI and simultaneously obtain imaging signals and display an image of the ROI during the therapy.

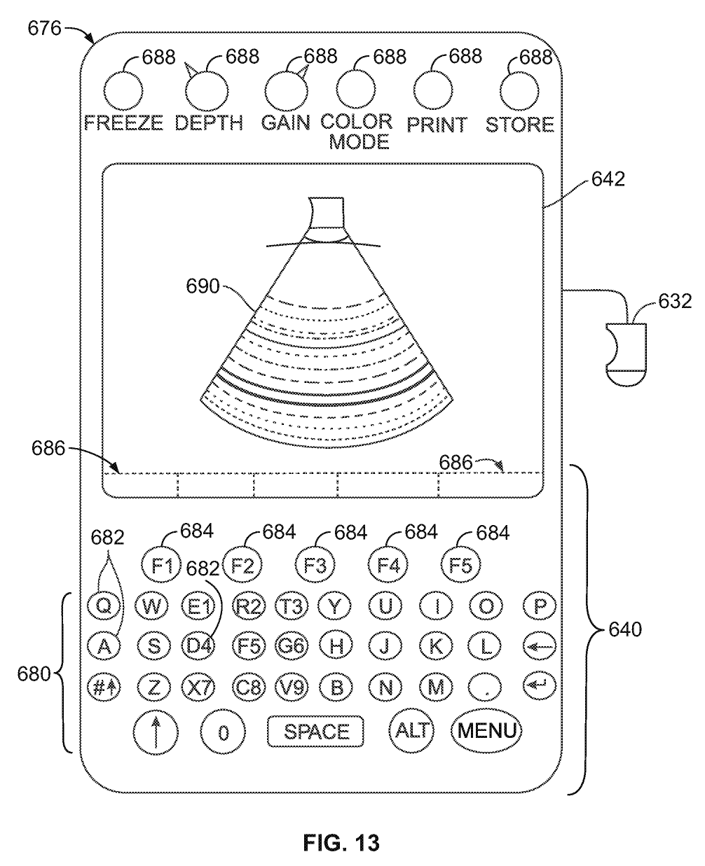

FIG. 13 shows another example of an ultrasound system and, in particular, a hand carried or pocket-sized ultrasound imaging system 676. In the system 676, a display 642 and a user interface 640 form a single unit. By way of example, the pocket-sized ultrasound imaging system 676 may be a pocket-sized or hand-sized ultrasound system approximately 2 inches wide, approximately 4 inches in length, and approximately 0.5 inches in depth and weighs less than 3 ounces. The display 642 may be, for example, a 320×320 pixel color LCD display (on which a medical image 690 may be displayed in combination with a graphical representation(s) as described above). A typewriter-like keyboard 680 of buttons 682 may optionally be included in the user interface 640. It should be noted that the various embodiments may be implemented in connection with a pocket-sized ultrasound system 676 having different dimensions, weights, and power consumption.

Multi-function controls 684 may each be assigned functions in accordance with the mode of system operation. Therefore, each of the multifunction controls 684 may be configured to provide a plurality of different actions. Label display spaces 686 associated with the multi-function controls 684 may be included as necessary on the display 642. The system 676 may also have additional keys and/or controls 688 for special purpose functions, which may include, but are not limited to “freeze,” “depth control,” “again control,” “color-mode,” “print,” and “store.”

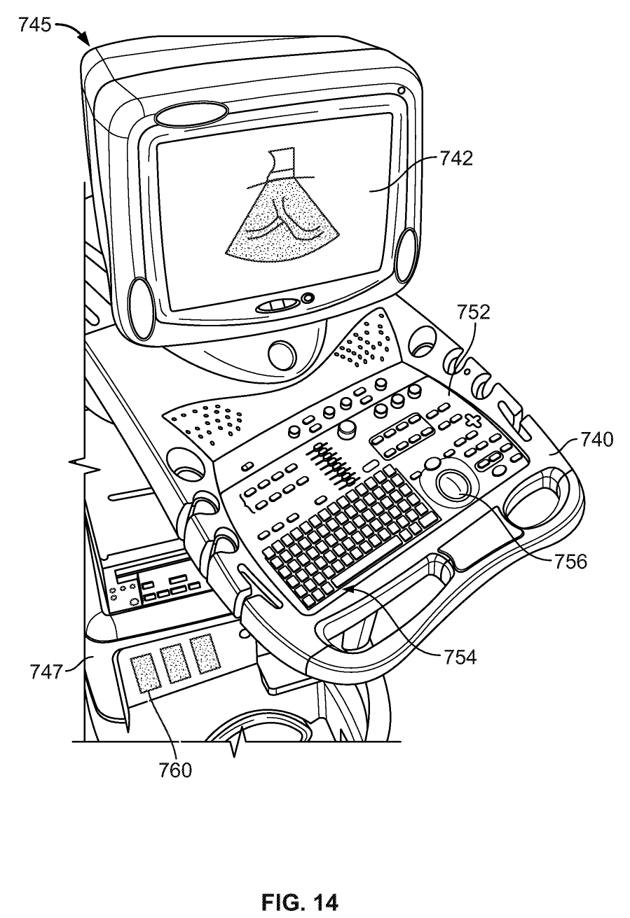

As another example shown in FIG. 14, a console-based ultrasound system 745 may be provided on a movable base 747 that may be configured to display the region of interest during a therapy session. The system 745 may also be referred to as a cart-based system. A display 742 and user interface 740 are provided and it should be understood that the display 742 may be separate or separable from the user interface 740. The user interface 740 may optionally be a touchscreen, allowing the operator to select options by touching displayed graphics, icons, and the like.

The user interface 740 also includes control buttons 752 that may be used to control the portable ultrasound imaging system 745 as desired or needed, and/or as typically provided. The user interface 740 provides multiple interface options that the user may physically manipulate to interact with ultrasound data and other data that may be displayed, as well as to enter user inputs and set and change imaging or therapy parameters. The interface options may be used for specific inputs, programmable inputs, contextual inputs, and the like. For example, a keyboard 754 and track ball 756 may be provided. The system 745 has at least one probe port 760 for accepting probes.

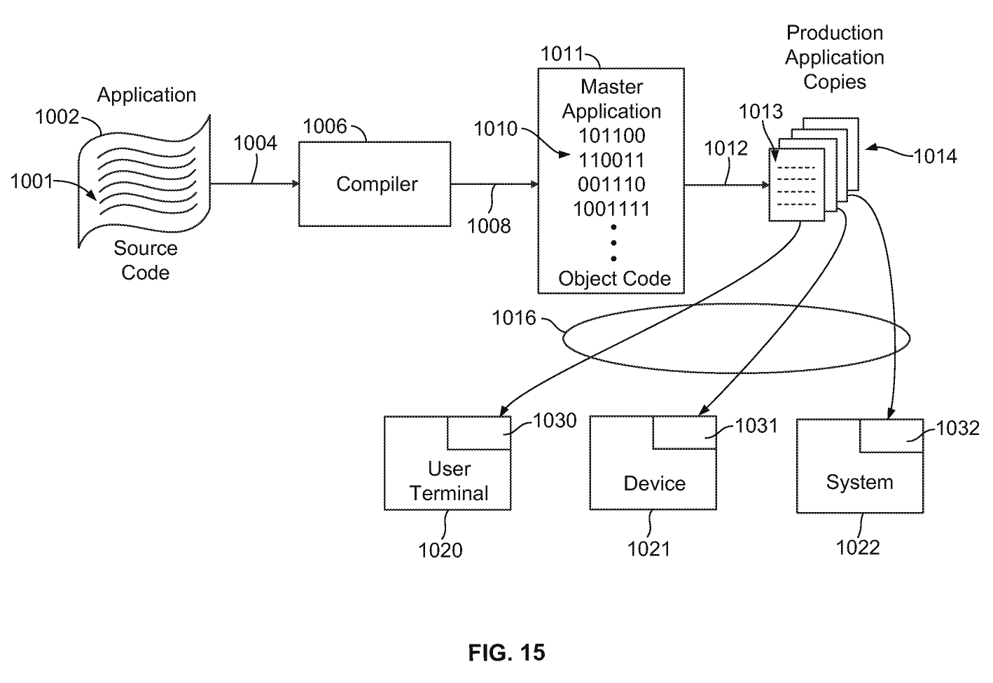

FIG. 15 is a block diagram of exemplary manners in which various embodiments described herein may be stored, distributed and installed on computer readable medium. In FIG. 15, the “application” represents one or more of the methods and process operations discussed above.

As shown in FIG. 15, the application is initially generated and stored as source code 1001 on a source computer readable medium 1002. The source code 1001 is then conveyed over path 1004 and processed by a compiler 1006 to produce object code 1010. The object code 1010 is conveyed over path 1008 and saved as one or more application masters on a master computer readable medium 1011. The object code 1010 is then copied numerous times, as denoted by path 1012, to produce production application copies 1013 that are saved on separate production computer readable medium 1014. The production computer readable medium 1014 is then conveyed, as denoted by path 1016, to various systems, devices, terminals and the like. In the example of FIG. 15, a user terminal 1020, a device 1021 and a system 1022 are shown as examples of hardware components, on which the production computer readable medium 1014 are installed as applications (as denoted by 1030-1032).

The source code may be written as scripts, or in any high-level or low-level language. Examples of the source, master, and production computer readable medium 1002, 1011 and 1014 include, but are not limited to, CDROM, RAM, ROM, Flash memory, RAID drives, memory on a computer system and the like. Examples of the paths 1004, 1008, 1012, and 1016 include, but are not limited to, network paths, the internet, Bluetooth, GSM, infrared wireless LANs, HIPERLAN, 3G, satellite, and the like. The paths 1004, 1008, 1012, and 1016 may also represent public or private carrier services that transport one or more physical copies of the source, master, or production computer readable medium 1002, 1011, or 1014 between two geographic locations. The paths 1004, 1008, 1012, and 1016 may represent threads carried out by one or more processors in parallel. For example, one computer may hold the source code 1001, compiler 1006 and object code 1010. Multiple computers may operate in parallel to produce the production application copies 1013. The paths 1004, 1008, 1012, and 1016 may be intra-state, inter-state, intra-country, inter-country, intra-continental, inter-continental and the like.

As used throughout the specification and claims, the phrases “computer readable medium” and “instructions configured to” shall refer to any one or all of i) the source computer readable medium 1002 and source code 1001, ii) the master computer readable medium and object code 1010, iii) the production computer readable medium 1014 and production application copies 1013 and/or iv) the applications 1030-1032 saved in memory in the terminal 1020, device 1021 and system 1022.

The various embodiments and/or components, for example, the monitor or display, or components and controllers therein, also may be implemented as part of one or more computers or processors. The computer or processor may include a computing device, an input device, a display unit, and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes described herein. The set of instructions may be in the form of a software program. The software may be in various forms such as system software or application software. Further, the software may be in the form of a collection of separate programs, a program module within a larger program or a portion of a program module. The software also may include modular programming in the form of object-oriented programming. The processing of input data by the processing machine may be in response to user commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

Although the embodiments described above are illustrated as treating adipose tissue, alternative embodiments may be used to treat other tissues within the body. For example, the above described embodiments may be used to image and treat a tumor within a region of interest. As described above with respect to adipose tissue, embodiments may be used to automatically identify the tumor and/or to allow user inputs to identify treatment spaces within a region of interest and to set therapy parameters for the treatment. Furthermore, embodiments described herein may be used for palliative treatments for cancer, thermal treatment of muscles, or ultrasonically activating drugs, proteins, stem cells, vaccines, DNA, and gene delivery.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. Dimensions, types of materials, orientations of the various components, and the number and positions of the various components described herein are intended to define parameters of certain embodiments, and are by no means limiting and are merely exemplary embodiments. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An ultrasound imaging and therapy system, comprising:
an ultrasound probe;
an ultrasound diagnostic module to control the probe to obtain diagnostic ultrasound signals from a region of interest (ROI), the ROI including adipose tissue and non-adipose tissue, the diagnostic module analyzing the diagnostic ultrasound signals and automatically differentiating adipose tissue from non-adipose tissue; and
an ultrasound therapy module to control the probe to deliver, during a therapy session, a therapy at a treatment location based on a therapy parameter to the adipose tissue differentiated by the ultrasound diagnostic module.

2. The system in accordance with claim 1 wherein the diagnostic module differentiates the adipose tissue by identifying a barrier between the adipose and non-adipose tissue.

3. The system in accordance with claim 1 wherein the diagnostic module differentiates the adipose tissue by directly measuring a plurality of points within the ROI for an adipose characteristic.

4. The system in accordance with claim 3 wherein the adipose characteristic constitutes one of a measure of tissue stiffness, longitudinal or shear wave velocity, density, cavitation inducibility, thermal conductivity, and specific heat.

5. The system in accordance with claim 1 wherein the diagnostic module differentiates the adipose tissue based on one of elastography and B-mode analysis of the diagnostic ultrasound signals.

6. The system in accordance with claim 1 wherein the diagnostic module obtains a first set of diagnostic ultrasound signals during an initial scan before the therapy module begins to deliver the therapy to the ROI and obtains a second set of diagnostic ultrasound signals during a supplemental scan during the therapy session, the therapy module adjusting the therapy parameter based on the second set of diagnostic ultrasound signals.

7. The system in accordance with claim 1 wherein the diagnostic module automatically sets the therapy parameter based on the diagnostic ultrasound signal obtained by the diagnostic module.

8. The system in accordance with claim 7 wherein the therapy parameter represents at least one of a focal region depth, a focal region size, an ablation time for each point, an energy level of the therapy, and a rate of focal region movement within the ROI during the therapy session.

9. The system in accordance with claim 1 wherein the diagnostic module identifies a thickness of a layer of the adipose tissue and the therapy module adjusts the therapy parameter based on the thickness.

10. The system in accordance with claim 1 wherein the diagnostic module analyzes the diagnostic ultrasound signals to confirm a location of the adipose tissue prior to delivering the therapy.

11. The system in accordance with claim 1 wherein the diagnostic module analyzes the diagnostic ultrasound signals to confirm the treatment of the treatment location.

12. A method for delivering therapy to a region of interest (ROI) in a patient, the method comprising:
obtaining diagnostic ultrasound signals from a region of interest (ROI), the ROI including adipose tissue and non-adipose tissue:
analyzing the diagnostic ultrasound signals and automatically differentiating adipose tissue from non-adipose tissue; and
delivering therapy, during a therapy session, at a treatment location based on a therapy parameter to the adipose tissue differentiated by the ultrasound diagnostic module.

13. The method in accordance with claim 12 wherein the automatically differentiating includes identifying a barrier between the adipose and non-adipose tissue.

14. The method in accordance with claim 12 wherein the automatically differentiating includes directly measuring a plurality of points within the ROI for an adipose characteristic.

15. The method in accordance with claim 14 wherein the adipose characteristic constitutes one of, a measure of tissue stiffness, longitudinal or shear wave velocity, density, cavitation inducibility, thermal conductivity and specific heat

16. The method in accordance with claim 12 wherein the automatically differentiating includes using one of elastography and B-mode analysis of the diagnostic ultrasound signals to automatically differentiate the adipose tissue from the non-adipose tissue.

17. The method in accordance with claim 12 wherein the obtaining diagnostic ultrasound signals from the ROI includes obtaining a first set of diagnostic ultrasound signals during an initial scan before delivering the therapy to the ROI and obtaining a second set of diagnostic ultrasound signals during a supplemental scan during the therapy session, the method further comprising adjusting the therapy parameter based on the second set of diagnostic ultrasound signals.

18. The method in accordance with claim 12 further comprising automatically setting the therapy parameter based on the diagnostic ultrasound signals obtained.

19. The method in accordance with claim 18 wherein the therapy parameter represents at least one of a focal region depth, a focal region size, an ablation time for each point, an energy level of the therapy, and a rate of focal region movement within the ROI during the therapy session.

20. The method in accordance with claim 12 further comprising identifying a thickness of a layer of the adipose tissue and adjusting the therapy parameter based on the thickness.

21. The method in accordance with claim 12 further comprising analyzing the diagnostic ultrasound signals to confirm a location of the adipose tissue prior to delivering the therapy.

22. The method in accordance with claim 12 further comprising analyzing the diagnostic ultrasound signals to confirm the treatment of the treatment location.
